(19) United States
(12) Plant Patent Application Publication
(10) Pub. No.: US 2007/0143899 P1
(43) Pub. Date: Jun. 21, 2007

(54) Chrysanthemum plant named 'Achilles'

(75) Inventor: Mark Roland Boeder, The Hague (NL)

Correspondence Address: STEPTOE & JOHNSON LLP, 1330 CONNECTICUT AVENUE, N.W., WASHINGTON, DC, 20036, US

(73) Assignee: Chrysanthemum Breeders Association N.V., Rijsenhout (NL)

(21) Appl. No.: 11/304,550

(22) Filed: Dec. 16, 2005

(51) Int. Cl. A01H 5/00

(52) U.S. Cl. PLT/286

(57) ABSTRACT

A chrysanthemum plant named ‘Achilles’ characterized by its medium sized blooms with yellow-bronze ray florets and prolific branching; natural season flowering date August 16; blooming for a period of 5 weeks.

### BACKGROUND OF THE INVENTION

‘Achilles’ is a product of a breeding and selection program for outdoor pot mums (garden mums) which had the objective of creating new chrysanthemum cultivars with a single, spoon type flower, a natural season flowering date around August 16; blooming for a period of 5 weeks. The new plant of the present invention comprises a new and distinct cultivar of chrysanthemum plant. ‘Achilles’ is a seedling resulting from a crossing program, set up by a previous breeder, and which records are unknown to the inventor. The new and distinct cultivar was discovered and selected as a flowering plant by Mark Roland Boeder on a cultivated field in Rijsenhout, The Netherlands in 2001. The first act of asexual production of ‘Achilles’ was accomplished when vegetative cuttings were taken from the initial selection in 2001 and propagated further in a controlled environment in Rijsenhout, The Netherlands. The new cultivar has been found to retain its distinctive characteristics through successive propagations.

### BRIEF DESCRIPTION OF THE DRAWINGS

The present invention of a new and distinct variety of chrysanthemum is shown in the accompanying drawings, the color being as nearly true as possible with color photographs of this type.

FIG. 1 shows a plant of the cultivar in full bloom.

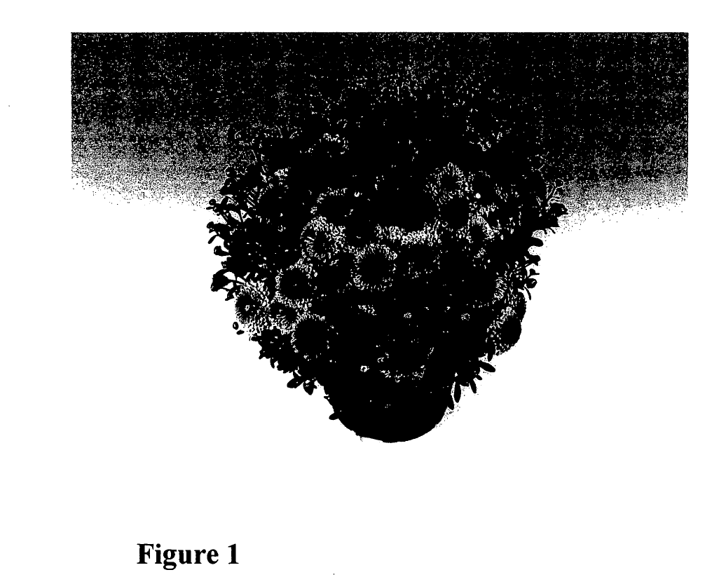

FIG. 2 shows the various stages of bloom of the new cultivar.

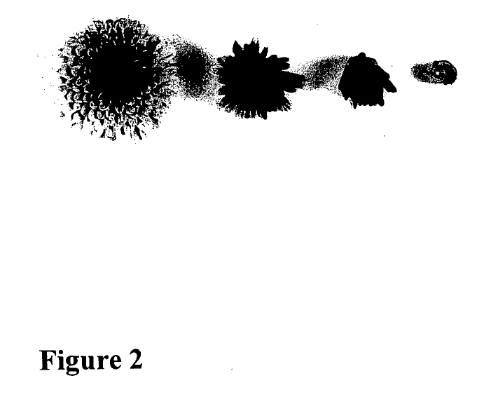

FIG. 3 shows the various stages of foliage of the new cultivar.

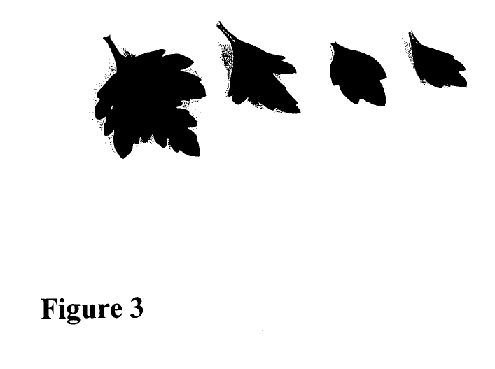

### DESCRIPTION OF THE INVENTION

This new variety of chrysanthemum is of the botanical classification Chrysanthemum morifolium L. The observations and measurements were gathered from plants grown out door in Rijsenhout, The Netherlands under natural day length and temperature and planted in week 24 in 2004. The natural blooming date of this crop was August 16-21 (week 34). The average height of the plants was 27 cm. No growth retardants were used. No tests were done on disease or insect resistance or susceptibility. No tests were done on cold or drought tolerance. This new variety produces medium sized blooms with yellow-bronze ray florets blooming for a period of 5 weeks.

From the cultivars known to inventor the most similar existing cultivar in comparison to ‘Achilles’ is ‘Mars’ (U.S. Plant Pat. No. 14,678). When ‘Mars’ and ‘Achilles’ are being compared the following differences are noticed: The differences of ‘Mars’ and ‘Achilles’ are (1). Natural flowering date; (2). Ray-floret type; and (3). Ray-floret color. (1) The natural season blooming of ‘Mars’ occurs later than that of ‘Achilles’. (2) Both the color designations and the colors depicted in the photographs are due to photographic tolerances. The color chart used in this description is: The Royal Horticultural Society Colour Chart, edition 1995.

### Table 1. Botanical Description of Chrysanthemum Plant ‘Achilles’

Bud:
   Size.—Small; cross-section 0.5 cm, height 0.5 cm.
   Outside color.—Yellow-green 145D.
   Involucral bracts.—2 rows, length 7 mm, width 3 mm.
   Involucral bracts among disc-florets.—Not present.
   Involucral bracts color.—Green 138A.
Bloom:
   Type.—Single, spoon.
   Height.—1.5 cm.
   Size.—Medium.
   Fully expanded.—4.5 cm.
   Peduncle length.—7 cm.
   Peduncle color.—Green 138B.
   Number of blooms per branch.—Approx. 8 blooms per branch.
   Performance on the plant.—5 weeks.
   Ray-floret margin.—Entire.
   Ray-floret length.—2 cm.
   Ray-floret width.—0.2-0.3 cm.
   Ratio length/width.—High.
   Shape of tip.—Rounded.
Disc florets:
   Disc diameter.—0.5-0.7 cm.
   Distribution of disc florets.—Numerous.
   Shape.—Tubular.
   Color.—Yellow 11D.
   Receptacle shape.—Domed raised.
Reproductive organs:
   Stamen.—Present in disc florets only.
   Stamen color.—Yellow-green 144A.
   Pollen.—Present.
   Pollen color.—Yellow 7A.
   Styles.—Thick.
   Style color.—Yellow 13A.
   Style length.—0.4 cm.
   Stigma color.—Yellow-green 144A.
   Stigma width.—1 mm.
   Ovaries.—Enclosed in calyx.
Plant:
   Form.—Grown as a spray type potmum, outdoor mounded and round.
   Growth habit.—Spherical shape.
   Growth rate.—Moderate.
   Height.—27 cm.
   Width.—25 cm.
   Stem color.—Yellow-green 147B with streaks of Greyed-red 182B.
   Stem strength.—Weak.
   Stem brittleness.—Brittle.
   Stem anthocyanin coloration.—Present.
   Internode length.—2.5 cm.
   Length of lateral branch.—From top to bottom 15-16 cm.
   Lateral branch color.—Yellow-green 147B-C.
   Lateral branch, attachment.—Medium strength.
   Branching (average number of lateral branches).—Good with 5-6 breaks after pinching.
   Natural season blooming date.—August 16-September 20.
Foliage:
   Leaf color.—Upper side Green 139 A-B Lower side Green 139C.
   Color midvein.—Upper side Yellow-green 147D Lower side Yellow-green 148C.
   Size.—Small; length 3-5 cm, width 2-3 cm.
   Quantity (number per lateral branch).—25-30.
   Shape.—Elliptic.
   Texture upper side.—Glabrous.
   Texture under side.—Pubescent.
   Venation arrangement.—Palmate.
   Shape of the margin.—Serrated.
   Shape of base of sinus between lateral lobes.—Rounded.
   Margin of sinus between lateral lobes.—Diverging.
   Shape of base.—Obtuse.
   Apex.—Mucronulate.
   Petiole length.—0.5-1 cm.
   Petiole color.—Yellow-green 147D.

TABLE 2
Differences with the comparison variety

| | ‘Achilles’ | ‘Mars’ |
|---|---|---|
| Natural season blooming date | August 16-21 | September 6-11 |
| Length corolla tube of ray-florets | 1.5 cm | 0.3 cm |
| Color upper surface ray-florets | Greyed-orange | Yellow-orange 22B |
| | 173C to 175C | |

I claim:

1. A new and distinct variety of chrysanthemum plant as described and illustrated.
